(19) United States
(12) Patent Application Publication
(10) Pub. No.: US 2020/0306335 A1
(43) Pub. Date: Oct. 1, 2020

(54) A Dietary Composition Comprising Plant-Based Sources of Fatty Acids

(71) Applicant: Caroline Sims, London (GB)

(72) Inventor: Caroline Sims, London (GB)

(21) Appl. No.: 16/759,716

(22) PCT Filed: Nov. 1, 2018
(86) PCT No.: PCT/GB2018/000141

§ 371 Date: Apr. 27, 2020

(30) Foreign Application Priority Data
Nov. 1, 2017 (GB) 1718084.5

(51) Int. Cl. A61K 36/889; A61K 31/20

(52) U.S. Cl. CPC A61K 36/889; A61K 31/20

(57) ABSTRACT

A dietary composition including saw palmetto and coconut oil in a proportion whereby the daily dose provides a controlled amount of fatty acids, having a minimum of 37.5 mg lauric acid and 16.44 mg myristic acid.

### FIELD OF INVENTION

The present invention relates to a dietary composition comprising plant-based sources of fatty acids. More especially, the invention relates to a dietary composition including saw palmetto and coconut oil. Moreover, the invention relates to such a dietary composition which provides controlled and standardised minimum quantities of myristic acid, as well as lauric acid.

### BACKGROUND OF THE INVENTION

Acne, hair loss, hirsutism and prostate conditions have been linked to high levels of Dihydrotestosterone (DHT) in the body. DHT is a chemical derivative of testosterone, created through the enzyme 5-alpha-reductase (5 alpha-R).

There are two types of 5-alpha-reductase (Types 1 and 2). Activity of Type 1 predominates in the sebaceous glands, where it influences sebum production, thus triggering acne. Type 2 is not linked to acne, but instead influences the hair follicles and prostate. Therefore, a mechanism to inhibit Type 1 5 alpha-R would offer a viable approach to the management of sebum production and, hence, acne. Mechanisms to inhibit Type 2 5 alpha-R are thought to be beneficial for hair loss, hirsutism and prostate conditions.

A study has shown that specific fatty acids inhibit either or both types of 5-alpha-reductase, thus preventing DHT synthesis. The active inhibitory fatty acids are: oleic acid, lauric acid, linoleic acid and myristic acid.

Oleic and linoleic acid are active inhibitors of 5 alpha-R Type 1, but have little to no inhibitory activity on 5 alpha-R Type 2. Lauric acid is an active inhibitor of both Type 1 and Type 2. Myristic acid was shown to strongly inhibit Type 2.

Saw palmetto berry has been shown to inhibit 5 alpha-reductase Type 1 and Type 2. This is thought to be due to its content of the above active fatty acids.

Commercial saw palmetto supplements are currently sold for male prostate conditions and less frequently for hair loss, as either oil (softgel) or powder capsules. More expensive production costs for oil capsules compared with powder capsules has resulted in higher upfront costs for businesses and less affordable prices for the end customer. Some companies have therefore chosen to provide saw palmetto in powder capsules. Oil capsules deliver 85-95% total fatty acids. Powdered saw palmetto provides up to 45-50% total fatty acids. As an example, this would equate to 80 mg of total fatty acid in 160 mg of saw palmetto powder, and 136 mg total fatty acid in the same amount of saw palmetto oil.

Current solutions though suffer from the problem of naturally varying quantities of lauric acid and myristic acid.

Saw Palmetto contains naturally varying amounts of lauric and myristic acid. Laboratory test results displaying the percentages of these fatty acids in different saw palmetto 45% extracts suggest that the variation across products each comprising 160 mg of one of these powder extracts, would be between 17.9 mg and 37.5 mg lauric acid, and 8.8 mg and 16.44 mg myristic acid. Oil extracts have also been found to vary significantly in their lauric and myristic acid content, displaying variations of 19% and 7% respectively.

Saw palmetto's fatty acids act differently on Type 1 and Type 2 5 alpha-R: oleic and linoleic acid are good inhibitors of Type 1 only, thus acting upon acne, and myristic acid was found to inhibit Type 2, thus acting upon hair loss, hirsutism and prostate conditions. Lauric acid inhibits both Type 1 and Type 2, thus acting upon acne, hair loss, hirsutism and prostate conditions. Lauric acid is thus the fatty acid with the broadest health benefits and yet, the naturally varying amounts of lauric and myristic acid renders products uncertain as to the actual quantities contained therein. This has the following consequences:
i) For prostate issues and hair loss, companies have paired saw palmetto with pumpkin seed oil or extract, which allows for control over the quantities of oleic and linoleic acid, but no such attempts have been made to identify and control the amount of lauric and myristic acid;
ii) Lauric and myristic acid are the only fatty acids in saw palmetto which inhibit Type 2 5 alpha-R, the type which influences prostate issues and hair loss. Oleic and linoleic acid display little to no activity on Type 2 5 alpha-R. Thus, the commercial saw palmetto products which add pumpkin seed oil/extract to target hair loss or prostate issues by inhibiting 5 alpha-R are in reality, targeting the wrong Type of 5 alpha-R and thus offering little added benefit in this context.

Due to the naturally varying amounts of lauric and myristic acid in saw palmetto, many prostate or hair loss products will be less effective than others, and also from batch to batch. In order to target hair loss and prostate issues more consistently and effectively, it is thus necessary to control the amounts of lauric and myristic acid and guarantee standardised minimum quantities in products comprising saw palmetto, which has not been attempted before.

Dietary products combining saw palmetto with other plant-based sources of fatty acids have never been sold for acne or skin health. If products were to be provided for skin care, due to the naturally varying amounts of lauric acid in saw palmetto, many of them would be less effective than others, and also from batch to batch. So far, no attempts have been made to prevent this variation. It would therefore be beneficial to control the amount of lauric acid and guarantee standardised minimum quantities in products comprising saw palmetto, so that these products can be sold and targeted for improving skin health.

Some companies have simply increased the dose of saw palmetto, to try to raise the fatty acid levels of saw palmetto products. However, industry recognised advice is to consume nutrients from a balanced range of food sources to ensure a larger variety of health benefits.

The present invention seeks to provide a dietary composition which provides controlled and standardised minimum quantities of myristic acid, as well as lauric acid, the fatty acid with the broadest health benefits.

The present invention enables controlled and standardised minimum amounts of lauric acid and myristic acid in products comprising saw palmetto, thereby reducing variation in product effectiveness. No such product exists in the current market and thus it significantly extends customer awareness and choice in the important areas of acne, skin care, hair loss, hirsutism and prostate related issues.

Coconut oil and its powdered version (coconut oil powder) have a similar fatty acid profile to saw palmetto and contain over 40% total fatty acids. Other than saw palmetto, coconut oil and coconut oil powder are the only plant-based sources of lauric and myristic acid and, in contrast with saw palmetto, coconut oil and coconut oil powder contain these two fatty acids in standardised minimum quantities. Therefore, coconut oil and coconut oil powder are necessary additions in order to enable controlled and standardised minimum quantities of lauric and myristic acid in products comprising saw palmetto.

Coconut oil and coconut oil powder contain the full range of fatty acids necessary for dual inhibition of both types of 5 Alpha-R, including lauric, myristic, oleic and linoleic acid.

Therefore, coconut oil is a preferred ingredient to complement saw palmetto in products targeting acne, hair loss, hirsutism or prostate conditions. Neither coconut oil or its powdered version have ever been combined with saw palmetto in a dietary composition before, and thus current products on the market have missed out on the unique and wide-ranging benefits of coconut oil and coconut oil-powder in enhancing saw palmetto's effects on improving acne, hair loss, hirsutism or prostate conditions through dual inhibition of 5 Alpha-R.

### STATEMENTS OF INVENTION

According to a first aspect of the invention there is provided, a dietary composition including saw palmetto and coconut oil in a proportion whereby the daily dosage provides a minimum of 37.5 mg lauric acid and 16.44 mg myristic acid, and wherein the daily dosage of the total composition provides a minimum of 80 mg total fatty acids.

Preferably, the daily dosage of the total composition provides a minimum of 136 mg total fatty acids.

Reference to daily dosage is not limited to one single consumption of the dietary composition. The dietary composition, or a selected amount of it, may be consumed on several occasions throughout the day, the daily dosage in which case being the total amount consumed in any one day.

Preferably, the composition includes saw palmetto in powder form.

Preferably, the composition includes coconut oil in powder form.

Preferably, the daily dosage provided by the proportion of saw palmetto and coconut oil provides a minimum of 136 mg total fatty acids.

### DETAILED DESCRIPTION OF AT LEAST ONE EMBODIMENT OF THE INVENTION

Example embodiments will now be described in detail.

These are provided for illustrative purposes only, they are not intended to be limiting in any way to the scope of the invention. Since numerous modifications and changes will readily occur to those skilled in the art, it is not desired to limit the invention to the exact construction and operation shown and described, and accordingly all suitable modifications and equivalents may be resorted to, falling within the scope of the invention. For example, the relative quantities of the ingredients may be varied to optimize the desired effects and/or additional ingredients may be added.

Within this specification, the terms “standardised minimum quantities” and “standardised minimum amounts” mean to guarantee a minimum amount of lauric and myristic acid in the composition and in this context, ensure the daily dose of the composition contains a lauric acid content of equal to or greater than 37.5 mg and a myristic acid content of equal to or greater than 16.44 mg.

Within this specification, “sunflower oil” and “sunflower oil powder” may include any of the following varieties: virgin, extra-virgin, organic, high oleic, high linoleic, mid-oleic or regular, water dispersible, cold water dispersible, or a combination thereof.

Within this specification, “coconut oil” and “coconut oil powder” may include any of the following varieties: organic, virgin, extra-virgin, cold water dispersible, and water dispersible.

Within this specification, the term “about” means plus or minus 10%.

Within this specification, the terms “50% oil powders”, “coconut oil powder 50%” and “sunflower oil powder 50%” refer to oil-powders which contain about 50% total fat, and/or about 50% of the original oil within the powder.

Within this specification, the terms “70% oil powders”, “coconut oil powder 70%” and “sunflower oil powder 70%” refer to oil-powders which contain about 70% total fat, and/or about 70% of the original oil within the powder.

Within this specification, the term “50% load” refers to oil-powders which have been converted from oil to powder and retain 50% of the original oil within the powder.

Within this specification, the term “70% load” refers to oil-powders which have been converted from oil to powder and retain 70% of the original oil within the powder.

Within this specification, “85% extract” refers to a commercial saw palmetto oil extract which has been standardised to contain 85-95% total fatty acids.

Within this specification, “45% extract” refers to a commercial saw palmetto powder extract which has been standardised to contain 45-50% total fatty acids.

Within this specification, “25% extract refers” to a commercial saw palmetto powder extract which has been standardised to contain 25% total fatty acids.

Within this specification, the term “hair loss” also includes the following: “alopecia”, “balding”, “thinning hair” and “pattern hair loss”.

Within this specification, the term “hirsutism” refers to abnormal or unwanted hair growth on a woman's face or body.

Within this specification, the term “prostate conditions” or “prostate issues” includes the following conditions and symptoms: frequent urination, urgency of urination, urinary hesitancy, weak urination, urinary blockage, urinary leakage, urinary dribbling, night urination, prostatitis, elevated prostate specific antigen levels, tumor cells, enlarged prostate, benign prostate hyperplasia, prostate cancer, and combinations thereof.

Within this specification, the term “PCOS” refers to Polycystic Ovary Syndrome.

Within this specification, the terms “treat” and “treating” refer to the reduction, slowing, stopping, limiting, prevention, and/or elimination of hair loss, acne, hirsutism, prostate conditions, hormone imbalance, and/or the effects of PCOS. The treatment may also improve skin and/or hair quality and texture as to minimize effects of acne or hair loss on appearance.

Within this specification, the terms “food supplement” and “dietary supplement” refer to concentrated sources of nutrients or other substances with a nutritional or physiological effect whose purpose is to supplement the normal diet. In other terms, food supplement means any food the purpose of which is to supplement the normal diet and which is a concentrated source of a vitamin or mineral or other substance with a nutritional or physiological effect, alone or in combination. A food supplement is preferably sold in dose form. The definition of ‘to supplement’ can be interpreted as taken in addition to the diet.

Within this specification, the singular form “a”, “an”, and “the” includes plural references unless indicated otherwise.

Within this specification, “Orgen-Zn” and “Orgen-Bio” refer to products which can be purchased from Orgenetics.

The composition and the examples below may be mixed together by conventional mixing equipment known in the art of pharmaceuticals and food supplements, and inserted, in dosage-sized quantities, into capsules or tablets for oral use. The capsules may be soft-gel capsules, vegetarian soft-gel capsules, hard 2-piece gelatin capsules or hard 2-piece vegetarian capsules made from hydroxypropyl methyl cellulose.

The composition may be in oil form. Preferably the saw palmetto oil will be an 85% extract.

Preferably, the composition will be in powder form. If encapsulating the composition in powder form, it is preferable to use microencapsulated oil powders.

The composition may include at least one other plant-based source of fatty acids. This other plant-based source of fatty acids may be at least one of the following, which may be in oil form or may be in powder form: sunflower seed oil, sunflower oil, olive oil, flax seed oil, evening primrose oil, pumpkin seed oil, sea buckthorn oil, blackcurrant seed oil, chia seed oil, borage oil, grape seed oil, hemp seed oil, perilla seed oil, safflower seed oil, sesame seed oil, cranberry seed oil, wheat germ oil, echium seed oil.

All powder-based example compositions below use 50% oil powders or 50% load oil powders; however, they may be replaced with 70% oil powders or 70% load oil powders, if so desired.

The composition and examples below may be mixed with acceptable carriers, additives, colours and excipients. Any suitable excipient may be used, including for example inert diluents, disintegrating agents, binding agents, flowing/anti-caking agent, lubricating agents, wetting agents, stabilizers, emulsifiers, buffers, sweetening agents, flavouring agents, bulking agents, preservatives and the like. Examples of bulking agents which may be used are rice flour or rice powder. Examples of flowing/anti-caking agents which may be used are magnesium stearate and/or silicon dioxide. The material used for the capsule shell in the following examples may be substituted with another suitable material.

The amount of lauric and myristic acid in the composition can be controlled and contained in standardised minimum amounts by, but not limited to, the following processes:

Process when Using Powders
Source a saw palmetto powder. A 45% extract is preferred;
Source a coconut oil powder (preferably 50%), which has a guaranteed minimum quantity of lauric and myristic acid, for example, minimums of 20% and 8%, respectively.
Send a 100 g sample of the saw palmetto powder to a fatty acid testing laboratory, such as Eurofins;
Based on the results showing the quantity of lauric and myristic acid in the saw palmetto powder, decide how much coconut oil powder and saw palmetto powder are required in order to provide minimums of 37.5 mg and 16.44 mg in the composition, respectively;
Include enough coconut oil powder and saw palmetto powder in the composition so that the minimum quantities of lauric and myristic acid in the composition are 37.5 mg and 16.44 mg respectively. The amount of coconut oil and/or saw palmetto in the composition can be varied to achieve higher quantities of lauric and myristic acid if required. If repeating this process more than once for the same final product, it is preferable to keep the amount of saw palmetto the same each time.

Process when Using Oils
Source a saw palmetto oil. An 85% extract is preferred;
Source a coconut oil (preferably virgin), which has a guaranteed minimum quantity of lauric and myristic acid, for example, minimums of 44% and 13%, respectively;
Send a 100 g sample of the saw palmetto oil to a fatty acid testing laboratory, such as Eurofins;
Based on the results showing the quantity of lauric and myristic acid in the saw palmetto oil, decide how much coconut oil and saw palmetto oil are required in order to provide minimums of 37.5 mg and 16.44 mg in the composition, respectively;
Include enough coconut oil and saw palmetto oil in the composition so that the minimum quantities of lauric and myristic acid in the composition are 37.5 mg and 16.44 mg respectively. The amount of coconut oil and/or saw palmetto in the composition can be varied to achieve higher quantities of lauric and myristic acid if required. If repeating this process more than once for the same final product, it is preferable to keep the amount of saw palmetto the same each time.

Another Alternative Process which can be Used for Both Oils and Powders:
Source a saw palmetto oil or powder.
Source a coconut oil or powder which has a guaranteed minimum quantity of lauric and myristic acid.
Send 100 g samples of both products to a fatty acid testing laboratory, such as Eurofins;
Based on the results showing the exact quantities of lauric and myristic acid in both the saw palmetto and coconut oil, decide how much additional lauric and myristic acid is required in order to provide minimums of 37.5 mg and 16.44 mg in the composition, respectively;
Include enough coconut oil/powder and saw palmetto oil/powder in the composition so that the minimum quantities of lauric and myristic acid in the composition are 37.5 mg and 16.44 mg respectively. The amount of coconut oil and/or saw palmetto in the composition can be varied to achieve higher quantities of lauric and myristic acid if required. If repeating this process more than once for the same final product, it is preferable to keep the amount of saw palmetto the same each time.

All example compositions below have the following 3 features in common: including saw palmetto and coconut oil, in a proportion whereby the daily dose provides a minimum of 37.5 mg lauric acid and 16.44 mg myristic acid, and wherein the daily dose of the total composition provides a minimum daily dosage of 80 mg total fatty acids.

Composition 1

In Composition 1, the ingredients are combined such that one capsule of the composition provides each ingredient in the quantities shown below in Table 1. The composition is formulated as 2-piece vegetarian size 00 capsules. Table 1 also demonstrates Composition 1 providing above 80 mg total fatty acids. Table 1 also shows how Composition 1 is able to control and standardise the amount of lauric and myristic acid to above 37.5 mg and 16.44 mg respectively.

TABLE 1

| | | | Amount of | Amount of | Amount of |
|---|---|---|---|---|---|
| | Amount | | fatty acids | lauric acid | myristic acid |
| | per capsule | | per capsule | per capsule | per capsule |
| Ingredient | | | | | |
| Saw Palmetto Powder (45% Extract) | 160 | mg | 80.88 mg | 25.95 mg | 11.33 mg |
| Sunflower Oil Powder 50% (High | 101.5 | mg | 52.78 mg | n/a | n/a |
| Oleic, Microencapsulated) | | | | | |
| Coconut Oil Powder 50% | 158 | mg | 82.32 mg | 37.98 mg | 15.23 mg |
| (Microencapsulated) | | | | | |
| Acerola Cherry Powder (25% Vitamin C) | 50 | mg | n/a | n/a | n/a |
| 10% Natural Beta-Carotene/Mixed | 11 | mg | n/a | n/a | n/a |
| Carotenoids (D. Salina Algae) | | | | | |
| Other Ingredients: | | | | | |
| Gluten free Rice Flour | 145 | mg | n/a | n/a | |
| Hyproxypropylmethyl Cellulose | 96 | mg | n/a | n/a | n/a |
| (capsule shell) | | | | | |

| | | |
|---|---|---|
| Total fatty | Total lauric | Total myristic |
| acids per capsule: | acid per capsule: | acid per capsule: |
| 215.98 mg | 63.93 mg | 26.56 mg |

Example Method of Administering Composition 1 to a Specific Population

Composition 1 in weights shown in Table 1 above can be administered to women as a treatment with a view to maintaining normal/improving skin or treating acne.

The serving size is two capsules per day, taken with food. The composition can be taken as long as needed. This composition is not recommended for pregnant, lactating or breastfeeding women. This composition can be taken as a food supplement or a medicine.

Composition 2

In Composition 2, the ingredients are combined such that one capsule of the composition provides each ingredient in the quantities shown below in Table 2. The composition is formulated as 2-piece vegetarian size 1 capsules. Table 2 also demonstrates Composition 2 providing above 80 mg total fatty acids. TABLE 2 also shows how Composition 2 is able to control and standardise the amount of lauric and myristic acid to above 37.5 mg and 16.44 mg respectively.

TABLE 2

| | | | Amount of | Amount of | | Amount of | |
|---|---|---|---|---|---|---|---|
| | Amount | | fatty acids | lauric acid | | myristic acid | |
| | per capsule | | per capsule | per capsule | | per capsule | |
| Ingredient | | | | | | | |
| Saw Palmetto Powder (45% extract) | 160 | mg | 81 mg | 25.95 | mg | 11.33 | mg |
| Coconut Oil Powder (50% load) | 175 | mg | 72 mg | 35 | mg | 14 | mg |
| Organic Sesbania (Agati) Extract | 5 | mg | n/a | n/a | | | |
| (Orgen-Bio) | | | | | | | |
| Other Ingredients: | | | | | | | |

| | | | | |
|---|---|---|---|---|
| Gluten free Rice Flour (bulking agent) | 60 | mg | n/a | n/a |
| Hyproxypropylmethyl Cellulose | | | n/a | n/a |
| (capsule shell) | | | | |

| | | |
|---|---|---|
| Total fatty | Total lauric | Total myristic |
| acids per capsule: | acid per capsule: | acid per capsule: |
| 153 mg | 60.95 mg | 25.33 mg |

Example Method of Administering Composition 2 to a Specific Population

Composition 2 can be administered to men and women as a treatment with a view to maintaining normal/improving hair or treating hair loss. The serving size is two capsules per day, taken with food. It can be taken as long as needed. The composition is not recommended for pregnant, lactating or breastfeeding women. This composition can be taken as a food supplement or a medicine.

Composition 3

In Composition 3, the ingredients are combined such that one capsule of the composition provides each ingredient in the quantities shown below in Table 3. The composition is formulated as 2-piece vegetarian size 0 capsules. Composition 3 provides a daily dose of above 80 mg total fatty acids. Table 3 also shows how Composition 3 is able to control and standardise the amount of lauric and myristic acid to above 37.5 mg and 16.44 mg respectively.

TABLE 3

| | | | Amount of | | Amount of | | Amount of | |
|---|---|---|---|---|---|---|---|---|
| | Amount | | fatty acids | | lauric acid | | myristic acid | |
| | per capsule | | per capsule | | per capsule | | per capsule | |
| Ingredient | | | | | | | | |
| Saw Palmetto Whole Berry Powder | 200 | mg | 26 | mg | 7 | mg | 4.7 | mg |
| Coconut Oil Powder (50%, | 160 | mg | 83.36 | mg | 38.46 | mg | 15.42 | mg |
| Microencapsulated) | | | | | | | | |
| Pumpkin Seed Oil Powder (50% load) | 75 | mg | 27 | mg | n/a | | n/a | |
| Organic Sesbania (Agati) Extract | 4 | mg | n/a | | n/a | | | |
| (Orgen-Bio) | | | | | | | | |
| Organic Guava Extract (Orgen-Zn) | 40 | mg | n/a | | n/a | | n/a | |
| Other Ingredients: | | | | | | | | |

| | | | | |
|---|---|---|---|---|
| Gluten free Rice Flour (bulking agent) | 21 | mg | n/a | n/a |
| Hyproxypropylmethyl Cellulose | 96 | mg | n/a | n/a |
| (capsule shell) | | | | |

| | | |
|---|---|---|
| Total fatty | Total lauric | Total myristic |
| acids per capsule: | acid per capsule: | acid per capsule: |
| 136.36 mg | 45.46 mg | 20.12 mg |

Example Method of Administering Composition 3 to a Specific Population

Composition 3 can be administered to men and women as a cosmetic treatment with a view to maintaining normal hair growth or treating hair loss.

Alternatively, Composition 3 can be administered to men with a view to supporting prostate function, maintaining a normal prostate or treating prostate conditions.

Alternatively, Composition 3 can be administered to men and women as a cosmetic treatment with a view to improving or maintaining normal skin, hair and nails.

In all populations, the serving size is 2-3 capsules per day, taken with food. It can be taken as long as needed. The composition is not recommended for pregnant, lactating or breastfeeding women. This composition can be taken as a food supplement or a medicine.

Composition 4

In Composition 4, the ingredients are combined such that one capsule of the composition provides each ingredient in the quantities shown below in Table 4. The composition is formulated as 2-piece vegetarian size 1 capsules. Table 4 also demonstrates Composition 4 providing above 80 mg total fatty acids.

TABLE 4 also shows how Composition 4 is able to control and standardise the amount of lauric and myristic acid to above 37.5 mg and 16.44 mg respectively.

TABLE 4

| | | | Amount of | | Amount of | | Amount of | |
|---|---|---|---|---|---|---|---|---|
| | Amount | | fatty acids | | lauric acid | | myristic acid | |
| | per capsule | | per capsule | | per capsule | | per capsule | |
| Ingredient | | | | | | | | |
| Saw Palmetto (45% extract) | 160 | mg | 84.8 | mg | 26.74 | mg | 10.6 | mg |
| Coconut Oil Powder (50% load) | 175 | mg | 72 | mg | 35 | mg | 14 | mg |
| Sunflower Oil Powder (50% load, | 60 | mg | 27 | mg | n/a | | | |
| High Oleic) | | | | | | | | |
| Other Ingredients: | | | | | | | | |

| | | | | |
|---|---|---|---|---|
| Gluten free Rice Flour (bulking agent) | 5 | mg | n/a | n/a |
| Hyproxypropylmethyl Cellulose | | | n/a | n/a |
| (capsule shell) | | | | |

| | | |
|---|---|---|
| Total fatty | Total lauric | Total myristic |
| acids per capsule: | acid per capsule: | acid per capsule: |
| 183.8 mg | 61.74 mg | 24.6 mg |

Example Method of Administering Composition 4 to a Specific Population

Composition 4 can be administered to men with a view to maintaining normal prostate, supporting prostate function or treating prostate conditions.

Composition 4 can alternatively be administered to men or women as a cosmetic treatment with a view to maintaining normal hair/hair growth, improving hair or treating hair loss.

Composition 4 can alternatively be administered to women as a cosmetic treatment with a view to maintaining normal skin/improving skin or treating acne.

Composition 4 can alternatively be administered to women as a cosmetic treatment with a view to treating hirsutism or normal facial and bodily hair growth.

Composition 4 can alternatively be administered to women with a view to treating one or more of the symptoms of Polycystic Ovary Syndrome.

Alternatively, Composition 4 can be administered to the general population, without giving details of a function, as a food supplement.

In all populations, the serving size is two capsules per day, taken with food. It can be taken as long as needed. This composition can be taken as a food supplement or a medicine. The composition is not recommended for use by pregnant or breastfeeding women.

Composition 5

In Composition 5, the ingredients are combined such that one capsule of the composition provides each ingredient in the quantities shown below in Table 5. The composition is formulated as soft-gelatine capsules. Table 5 also demonstrates Composition 5 providing above 80 mg total fatty acids. TABLE 5 also shows how Composition 5 is able to control and standardise the amount of lauric and myristic acid to above 37.5 mg and 16.44 mg respectively.

TABLE 5

| | | Amount of | | Amount of | Amount of | |
|---|---|---|---|---|---|---|
| | Amount | fatty acids | | lauric acid | myristic acid | |
| Ingredient | per capsule | per capsule | | per capsule | per capsule | |
| Saw Palmetto Oil (85-95% fatty | 117 mg | 105 | mg | 37 mg | 17.56 | mg |
| acids) | | | | | | |
| Coconut Oil (Organic, Virgin) | 50 mg | 49.7 | mg | 25 mg | 8 | mg |

| | | |
|---|---|---|
| Total fatty | Total lauric | Total myristic |
| acids per capsule: | acid per capsule: | acid per capsule: |
| 154.7 mg | 62 mg | 25.56 mg |

Example Method of Administering Composition 5 to a Specific Population

Composition 5 can be administered to men with a view to maintaining a normal prostate or treating prostate conditions.

Composition 5 can alternatively be administered to men or women as a cosmetic treatment with a view to maintaining normal hair or treating hair loss.

Composition 5 can alternatively be administered to women as a cosmetic treatment with a view to treating hirsutism or normal facial and bodily hair growth.

Alternatively, Composition 5 can be administered to the general population, without giving details of a function, as a food supplement.

In all populations, the serving size is two capsules per day, taken with food. It can be taken as long as needed. This composition can be taken as a food supplement or a medicine. The composition is not recommended for use by pregnant or breastfeeding women.

Composition 6

In Composition 6, the ingredients are combined such that one capsule of the composition provides each ingredient in the quantities shown below in Table 6. The composition is formulated as 2-piece vegetarian size 0 capsules. The Acerola cherry can be replaced by 30 mg of Orgen-Zn (Organic Guava Extract). Table 6 also demonstrates Composition 6 providing above 80 mg total fatty acids. TABLE 6 also shows how Composition 6 is able to control and standardise the amount of lauric and myristic acid to above 37.5 mg and 16.44 mg respectively.

TABLE 6

| | | Amount of | Amount of | Amount of |
|---|---|---|---|---|
| | Amount | fatty acids | lauric acid | myristic acid |
| | per capsule | per capsule | per capsule | per capsule |
| Ingredient | | | | |
| Saw Palmetto Powder (45% extract) | 95 mg | 49 mg | 22 mg | 9.8 mg |
| Coconut Oil Powder (50% Load) | 95 mg | 39 mg | 19 mg | 7.6 mg |
| Blackcurrant Seed Oil Powder (50% Load) | 280 mg | 118 | n/a | n/a |
| Acerola Cherry Powder (25% vitamin c) | 30 mg | n/a | n/a | |
| Other Ingredients: | | | | |
| Hyproxypropylmethyl Cellulose | 96 mg | n/a | n/a | |
| (capsule shell) | | | | |
| | | Total fatty | Total lauric | Total myristic |
| | | acids per capsule: | acid per capsule: | acid per capsule: |
| | | 206 mg | 41 mg | 17.4 mg |

Example Method of Administering Composition 6 to a Specific Population

Composition 6 can be administered to men or women as a cosmetic treatment to maintain normal skin/improving skin or treating acne.

Composition 6 can alternatively be administered to men with a view to maintaining normal prostate or treating prostate conditions.

Composition 6 can alternatively be administered to men or women with a view to maintaining normal hair or treating hair loss.

Composition 6 can alternatively be administered to women as a cosmetic treatment of hirsutism or normal facial and bodily hair growth.

Alternatively, Composition 6 can be administered as a cosmetic treatment as a skin and hair supplement; or a skin, hair and nails supplement.

Composition 6 can alternatively be administered to women with a view to treating one or more of the symptoms of Polycystic Ovary Syndrome.

In all populations, the serving size of Composition 6 is two capsules per day, taken with food. It can be taken as long as needed. This composition can be taken as a food supplement or a medicine. The composition is not recommended for use by pregnant or breastfeeding women.

In Composition 7, the ingredients are combined such that one capsule of the composition provides each ingredient in the quantities shown below in Table 7. The composition is formulated as vegetarian soft-gel capsules. Table 7 also demonstrates Composition 7 providing above 80 mg total fatty acids. TABLE 7 also shows how Composition 7 is able to control and standardise the amount of lauric and myristic acid to above 37.5 mg and 16.44 mg respectively.

TABLE 7

| | | Amount of | | Amount of | | Amount of |
|---|---|---|---|---|---|---|
| | Amount | fatty acids | | lauric acid | | myristic acid |
| Ingredient | per capsule | per capsule | | per capsule | | per capsule |
| Saw Palmetto Oil (85-95% fatty | 80 mg | 71.8 | mg | 25.3 | mg | 12 mg |
| acids) | | | | | | |
| Sunflower Oil (Mid-Oleic) | 20 mg | 20 | mg | n/a | | n/a |
| Coconut Oil (Virgin) | 50 mg | 49.7 | mg | 25 | mg | 8 mg |

| | | | | |
|---|---|---|---|---|
| Acerola Juice Concentrate | 70 mg | n/a | n/a | n/a |
| Carrot Juice Concentrate | 50 mg | n/a | n/a | n/a |
| | | Total fatty | Total lauric | Total myristic |
| | | acids per capsule: | acid per capsule: | acid per capsule: |
| | | 141.5 mg | 50.3 mg | 20 mg |

Example Method of Administering Composition 7 to a Specific Population

Composition 7 in weights shown in Table 7 above can be administered to as a cosmetic treatment women with a view to maintaining normal skin or treating acne. The serving size is two capsules per day, taken with food. The composition can be taken as long as needed. This composition is not recommended for pregnant, lactating or breastfeeding women. This composition can be taken as a food supplement or a medicine.

Any of example compositions 1-7 can be administered with a view to inhibiting 5-alpha reductase and/or reducing DHT.

Any of example compositions 1-7 can be administered with a view to balancing hormones and/or treating hormonal imbalances.

It should be understood that various changes and modifications to the presently preferred embodiments described herein will be apparent to those skilled in the art.

1. A dietary composition including saw palmetto and coconut oil in a proportion whereby the daily dosage provides a minimum of 37.5 mg lauric acid and 16.44 mg myristic acid, and wherein the daily dosage of the total composition provides a minimum of 80 mg total fatty acids.

2-56. (canceled)
